(19) United States
(12) Patent Application Publication
(10) Pub. No.: US 2024/0190804 A1
(43) Pub. Date: Jun. 13, 2024

(54) Diruthenium Catalyst Compositions and Synthetic Processes Related Thereto

(71) Applicant: Emory University, Atlanta, GA (US)

(72) Inventors: Huw M. L. Davies, Suwanee, GA (US); Joshua Sailer, Lewisville, NC (US); Jack C. Sharland, Mendham, NJ (US); Bo Wei, Atlanta, GA (US)

(21) Appl. No.: 18/276,736

(22) PCT Filed: Feb. 10, 2022
(86) PCT No.: PCT/US2022/015912

§ 371 Date: Aug. 10, 2023

Related U.S. Application Data
(60) Provisional application No. 63/147,800, filed on Feb. 10, 2021.

(51) Int. Cl. C07C 67/343; B01J 31/22; C07F 15/00

(52) U.S. Cl. CPC C07C 67/343; B01J 31/223; C07F 15/0053; B01J 2231/4283; B01J 2531/0219; B01J 2531/821; C07C 2531/22; C07C 2601/02; C07C 2601/14

(57) ABSTRACT

This disclosure relates to compositions comprising diruthenium catalysts and uses related thereto. In certain embodiments, the diruthenium catalyst comprises a cyclopropyl ring substituted with a carboxylic acid ligand. In certain embodiments, the diruthenium catalyst comprises an N-(sulfonyl)pyrrolidine ring substituted with a carboxylic acid ligand. In certain embodiments, the diruthenium catalyst comprises a 2-(1,3-dioxoisoindolin-2-yl)acetic acid ligand. In certain embodiments, this disclosure relates to methods of using catalysts in chemical transformations disclosed herein.

### CROSS-REFERENCE TO RELATED APPLICATIONS

This application claims the benefit of U.S. Provisional Application No. 63/147,800 filed Feb. 10, 2021. The entirety of this application is hereby incorporated by reference for all purposes.

### STATEMENT REGARDING FEDERALLY SPONSORED RESEARCH OR DEVELOPMENT

This invention was made with government support under CHE1956154 and CHE1700982 awarded by the National Science Foundation. The government has certain rights in the invention.

### BACKGROUND

The cyclopropane ring is a common structural motif incorporated into many pharmaceutical agents. Elaborate chiral cyclopropanes have been incorporated into therapeutic scaffolds, such as the trisubstituted cyclopropanes in beclabuvir, paritaprevir, and glecaprevir. In these cases, three substituents are placed in a defined orientation. The syntheses of these cyclopropanes are challenging because they contain two stereogenic centers which are to be generated in a diastereoselective and enantioselective manner. Thus, there is a need to identify improved synthetic processes.

Davies et al. report asymmetric cyclopropanations by rhodium(II) N-(arylsulfonyl)prolinate catalyzed decomposition of vinyldiazomethanes in the presence of alkenes. J. Am. Chem. Soc. 1996, 118, 6897-6907.

Saha et al. report evaluation of a pair of diruthenium(I) catalysts for carbene-transfer reactions from ethyl diazoacetate. Organometallics, 2011, 30, 2051-2058.

Chepiga et al. report a guide to enantioselective dirhodium(II)-catalyzed cyclopropanation with aryldiazoacetates. Tetrahedron, 2013, 69, 5765-5771. See also U.S. Pat. No. 8,975,428.

Miyazawa et al. report chiral paddle-wheel diruthenium complexes for asymmetric catalysis. Nature Catalysis, 2020, 3, 851-858.

References cited herein are not an admission of prior art.

### SUMMARY

This disclosure relates to compositions comprising diruthenium catalysts and uses related thereto. In certain embodiments, the diruthenium catalyst comprises a cyclopropyl ring substituted with a carboxylic acid ligand. In certain embodiments, the diruthenium catalyst comprises an N-(sulfonyl)pyrrolidine ring substituted with a carboxylic acid ligand. In certain embodiments, the diruthenium catalyst comprises a 2-(1,3-dioxoisoindolin-2-yl)acetic acid ligand. In certain embodiments, this disclosure relates to methods of using catalysts in chemical transformations disclosed herein.

In certain embodiments, the disclosure relates to compositions comprising a catalyst of the following formula,

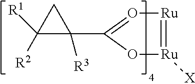

or derivatives or salts thereof wherein, R¹, R², R³ and X are defined herein.

In certain embodiments, the disclosure relates to compositions comprising Ru₂(S-p-Ph-TPCP)₄ X, Ru₂(S-p-Br-TPCP)₄ X, Ru₂(S-2Cl-5Br-TPCP)₄X, Ru₂(R-tris(p-^{t}BuC₆H₄)-TPCP)₄X, or a catalyst of formula

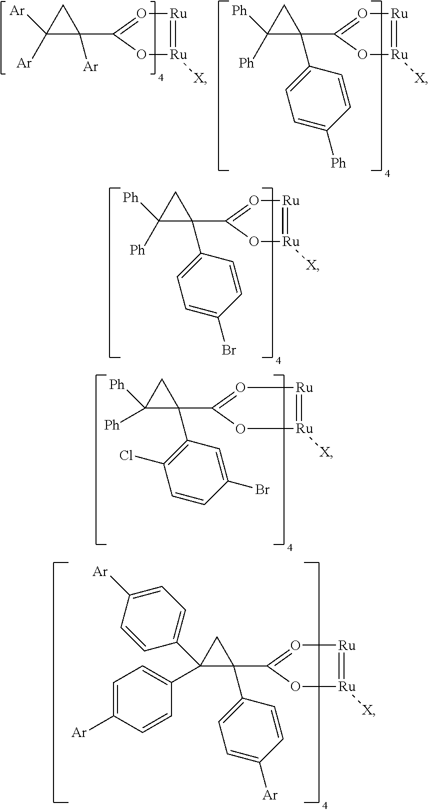

or derivatives or salts thereof wherein, Ar and X are defined herein, e.g., X is a covalently bound ligand, such as Cl, or a counterion such as PF₅⁻ or BARF⁻.

In certain embodiments, the disclosure relates to compositions comprising Ru₂(R-DOSP)₄ X, or a catalyst of formula

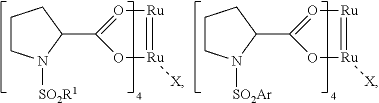

or derivatives or salts thereof wherein, R¹ and X are defined herein, e.g., X is a covalently bound ligand, such as Cl, or a counterion such as PF₅⁻ or BARF⁻.

In certain embodiments, Ar is p-alkyl-C₆H₄—.

In certain embodiments, the disclosure relates to compositions comprising Ru₂(S-TCPTTL)₄ X, Ru₂(S-PTAD)₄ X, Ru₂(S-TCPTAD)4 X or a catalyst of formula

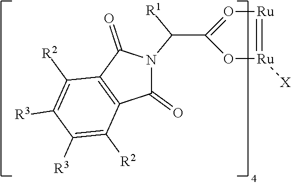

or derivatives or salts thereof wherein, R¹, R², R³, and X are defined herein.

In certain embodiments, the disclosure relates to methods of preparing diruthenium catalysts or intermediates disclosed herein by mixing starting materials with catalysts under conditions such that the catalysts or intermediates are formed.

In certain embodiments, the disclosure contemplates enantioselective reactions of donor/acceptor carbenoids, such as cyclopropanations, formal [4+3] cycloadditions, C—H functionalizations, and ylide transformations comprising mixing a compound comprising a carbenoid precursor, e.g., a diazo compound, and catalysts disclosed herein and reactive compounds under conditions such that a synthetic compound is formed.

### BRIEF DESCRIPTION OF THE DRAWINGS

FIG. 1A illustrates the proposed chemical structure of Ru₂(R-p-BrTPCP)₄; [Tetrakis[(R)-(−)-[(1R)-1-(4-bromophenyl)-2,2-diphenylcyclopropanecarboxylato] diruthenium(II/III) with a tetrakis(3,5-bis(trifluoromethyl)phenyl)borate (BArF) counter anion.

FIG. 1B illustrates the proposed chemical structure of Ru₂(R-p-BrTPCP)₄Cl; [Tetrakis[(R)-(−)-[(1R)-1-(4-bromophenyl)-2,2-diphenylcyclopropanecarboxylato] diruthenium(II/III)]chloride.

FIG. 1C illustrates the proposed chemical structure of Ru₂(R-p-PhTPCP)₄BArF; [Tetrakis[(R)-(−)-[(1R)-1-(4-phenyl(phenyl))-2,2-diphenylcyclopropanecarboxylato] diruthenium (II/III)] with a tetrakis(3,5-bis(trifluoromethyl)phenyl)borate counter anion.

FIG. 1D illustrates the proposed chemical structure of Ru₂(R-p-PhTPCP)₄Cl; [Tetrakis[(R)-(−)-[(1R)-1-(4-phenyl(phenyl))-2,2-diphenylcyclopropanecarboxylato] diruthenium(II/III)]chloride.

FIG. 1E illustrates the proposed chemical structure of Ru₂(S-2Cl5BrTPCP)₄BArF; [Tetrakis[(S)-1-(5-bromo-2-chlorophenyl)-2,2-diphenylcyclopropanecarboxylato] diruthenium(II/III)] with a tetrakis(3,5-bis(trifluoromethyl)phenyl)borate counter anion.

FIG. 1F illustrates the proposed chemical structure of Ru₂(S-2Cl5BrTPCP)₄Cl; [Tetrakis[(S)-(5-bromo-2-chlorophenyll)-2,2-diphenylcyclopropanecarboxylato] diruthenium(II/III)]chloride.

FIG. 1G illustrates the proposed chemical structure of Ru₂(R-tris(p-tBuC₆H₄)-TPCP)₄BArF; Tetrakis[(R)-1,2,2-tris[4′-(tert-butyl)-(1,1′-biphenyl]-4-yl]cyclopropane-1-carboxylato] diruthenium(II/III)] with a tetrakis(3,5-bis(trifluoromethyl)phenyl)borate counter anion.

FIG. 1H illustrates the proposed chemical structure of Ru₂(R-tris(p-tBuC₆H₄)-TPCP)₄Cl; Tetrakis [(R)-1,2,2-tris[4″-(tert-butyl)-(1,1′:4′,1″-terphenyl]-4-yl]cyclopropane-1-carboxylato] diruthenium(II/III)]chloride.

FIG. 1I illustrates the proposed chemical structure of Ru₂(R-3,5-di(p-tBuC₆H₄)-TPCP)₄BArF; Tetrakis [(R)-1-(4,4″-di-tert-butyl-[1,1′:3′,1″-terphenyl]-5′-yl)-2,2-diphenylcyclopropanecarboxylato] diruthenium(II/III)] with a tetrakis(3,5-bis (trifluoromethyl)phenyl)borate counter anion.

FIG. 1J illustrates the proposed chemical structure of Ru₂(R-3,5-di(p-tBuC₆H₄)-TPCP)₄Cl; Tetrakis [(R)-1-(4,4″-di-tert-butyl-[1,1′:3′,1″-terphenyl]-5′-yl)-2,2-diphenyl cyclopropanecarboxylato]diruthenium(II/III)]chloride.

FIG. 2A illustrates the proposed chemical structure of Ru₂(S-DOSP)₄BArF; [Tetrakis[(S)-(−)-N-(p-dodecylphenylsulfonyl)prolinato]diruthenium(II/III)] with a tetrakis(3,5-bis(trifluoromethyl)phenyl)borate counter anion.

FIG. 2B illustrates the proposed chemical structure of Ru₂(S-DOSP)₄Cl; [Tetrakis[(S)-(−)-N-(p-dodecylphenylsulfonyl)prolinato]diruthenium(II/III)chloride.

FIG. 3A illustrates the proposed chemical structure of Ru₂(S-TPPTTL)₄BArF [Tetrakis[N-tetraphenylphthaloyl-(S)-tert-leucinato]diruthenium(II/III)] with a tetrakis(3,5-bis (trifluoromethyl)phenyl)borate counter anion.

FIG. 3B illustrates the proposed chemical structure of Ru₂(S-TPPTTL)₄Cl; [Tetrakis[N-tetraphenylphthaloyl-(S)-tert-leucinato]diruthenium(II/III)]chloride.

FIG. 3C illustrates the proposed chemical structure of Ru₂(S-TCPTAD)₄BArF; [Tetrakis[(S)-(+)-(1-adamantyl)-(N-tetrachlorophthalimido)acetato]diruthenium(II/III)] with a tetrakis(3,5-bis(trifluoromethyl)phenyl)borate counter anion.

FIG. 3D illustrates the proposed chemical structure of Ru₂(S-TCPTAD)₄Cl; [Tetrakis[(S)-(+)-(1-adamantyl)-(N-tetrachlorophthalimido)acetato]diruthenium(II/III)]chloride.

FIG. 3E illustrates the proposed chemical structure of Ru₂(S-PTAD)₄BArF; [Tetrakis[(S)-(+)-(1-adamantyl)-(N-phthalimido)acetato]diruthenium(II/III)] with a tetrakis(3,5-bis(trifluoromethyl)phenyl)borate counter anion.

FIG. 3F illustrates the proposed chemical structure of Ru₂(S-PTAD)₄Cl; [Tetrakis[(S)-(+)-(1-adamantyl)-(N-phthalimido)acetato]diruthenium(II/III)]chloride.

FIG. 4 illustrates the use of diruthenium complexes disclosed herein in the synthesis of intermediates for the production pharmaceutical products such as beclabuvir.

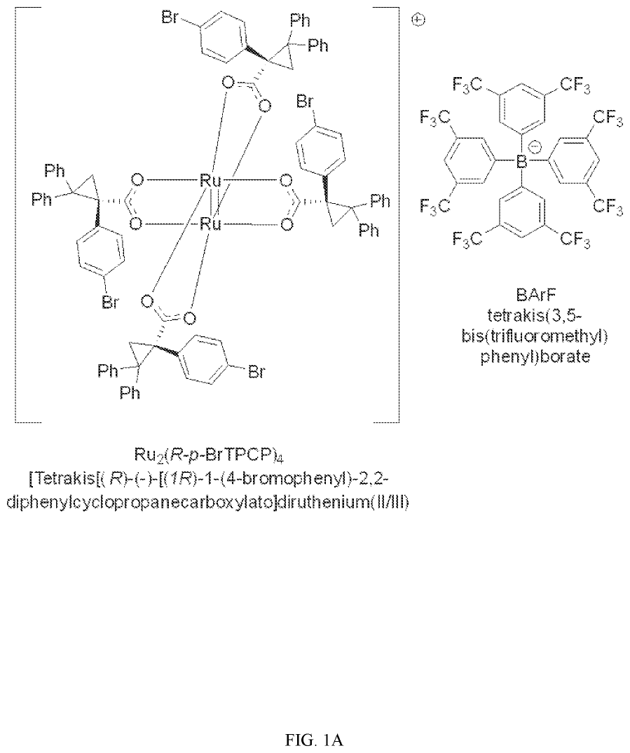

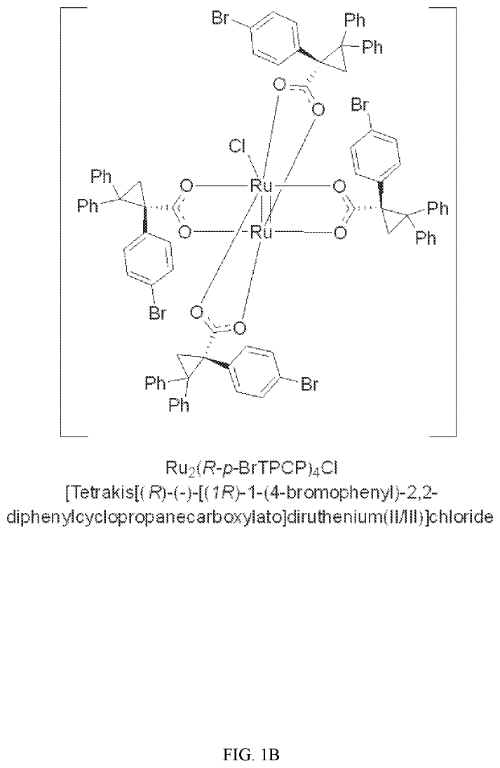

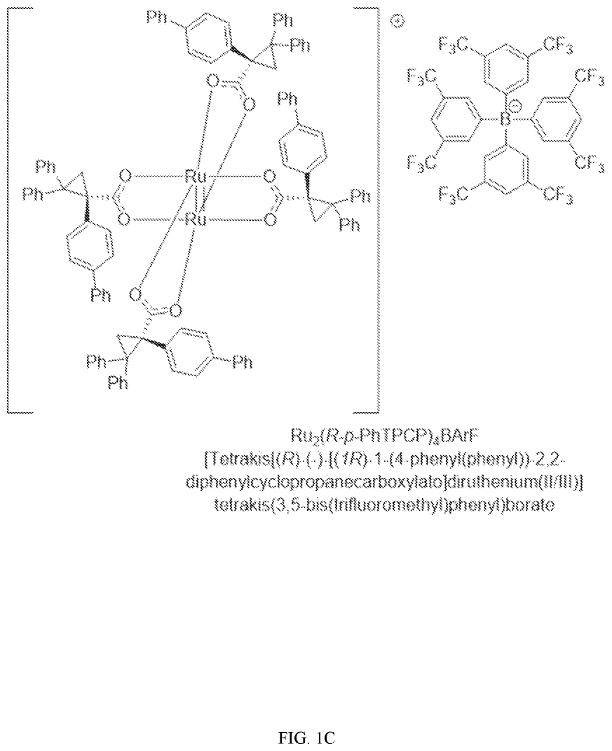

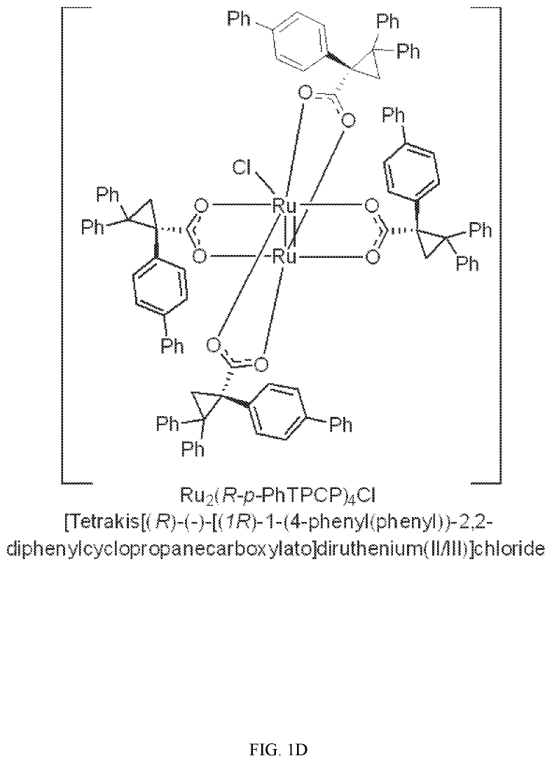

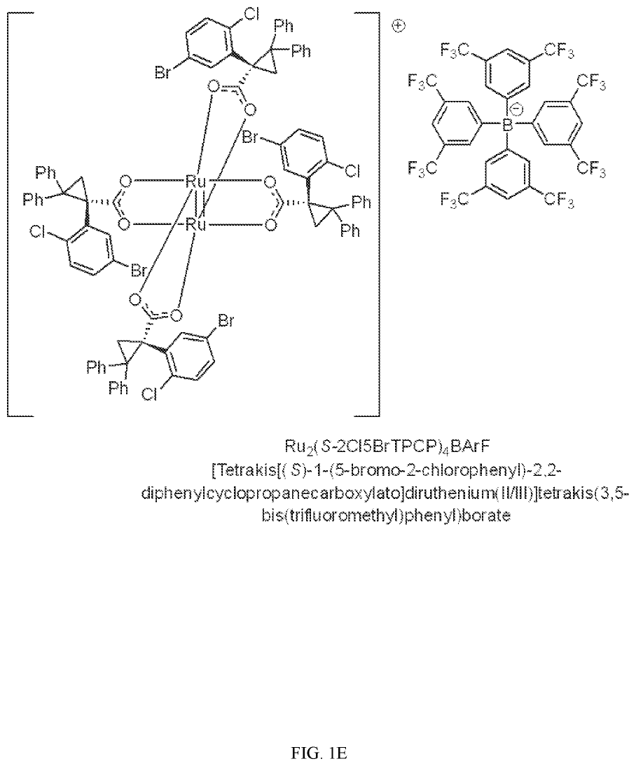

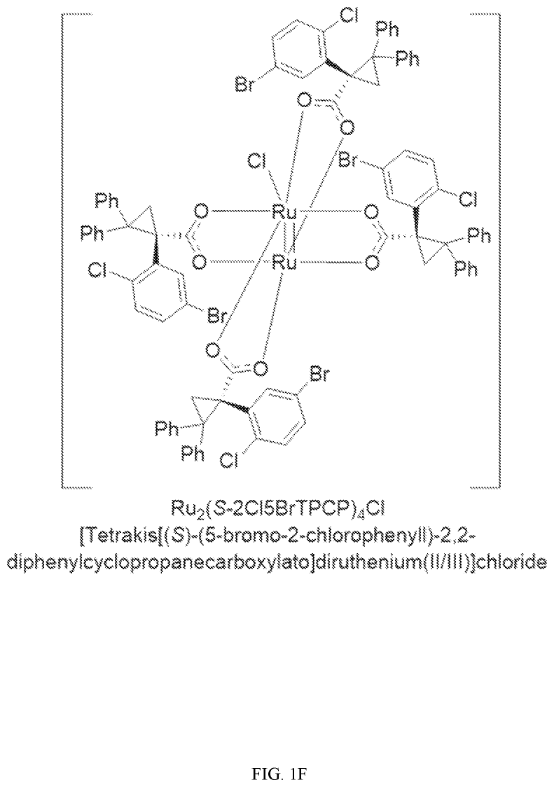

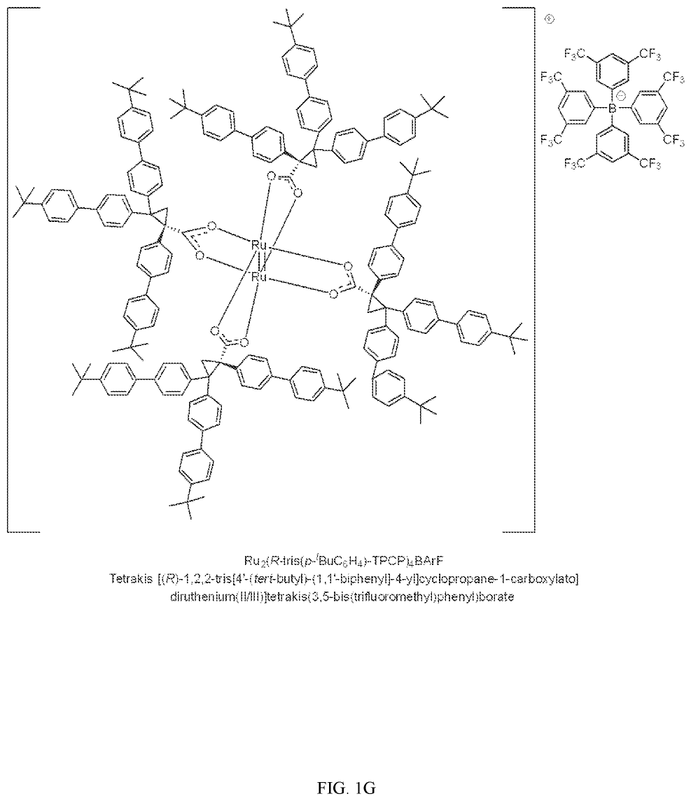

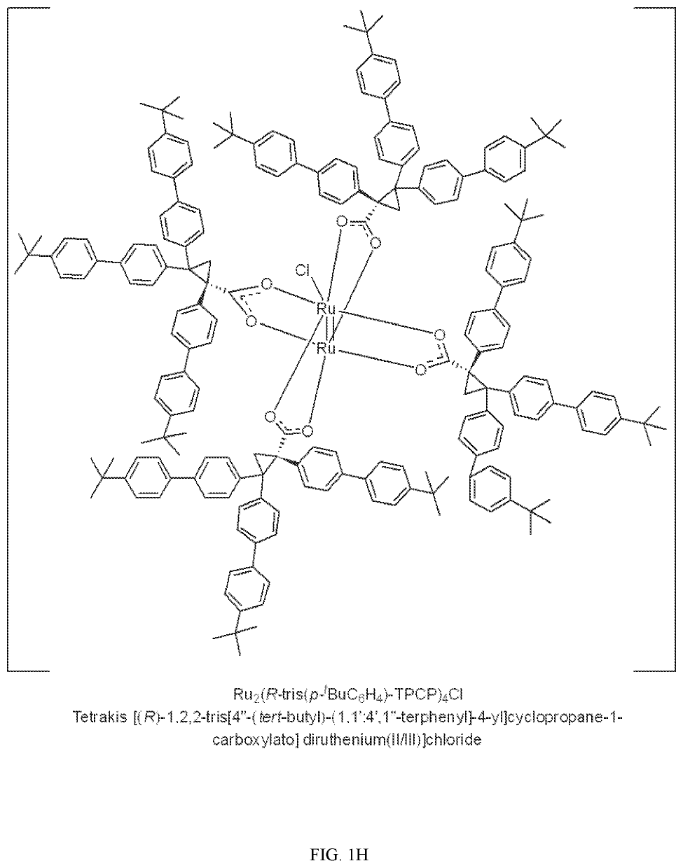

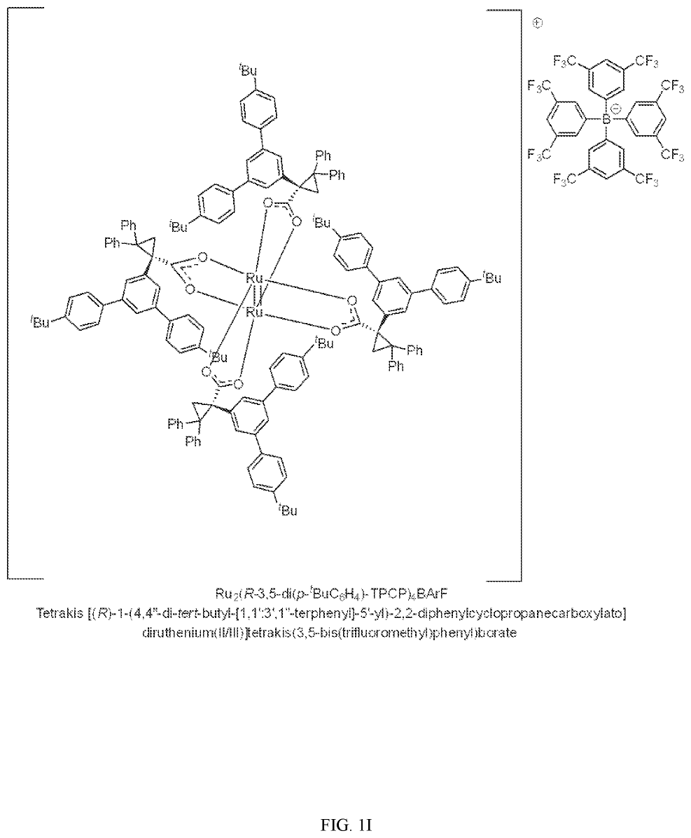

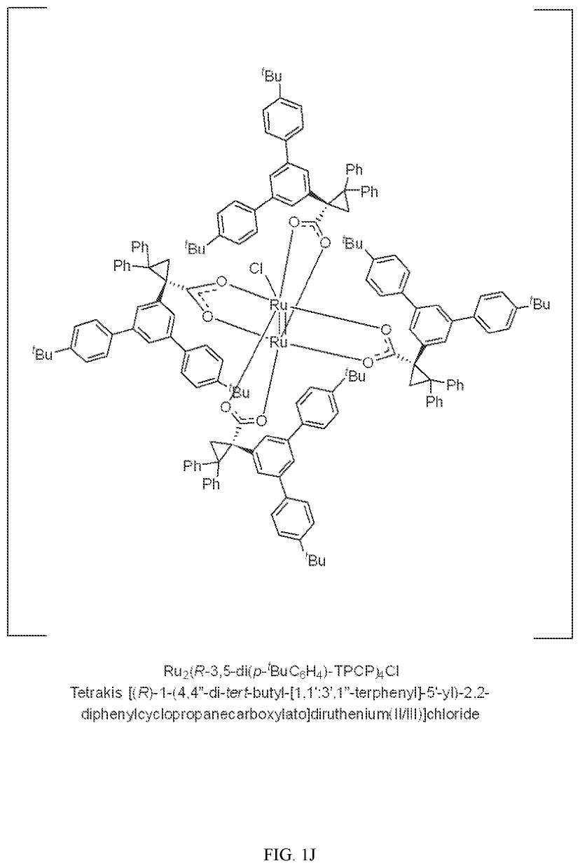

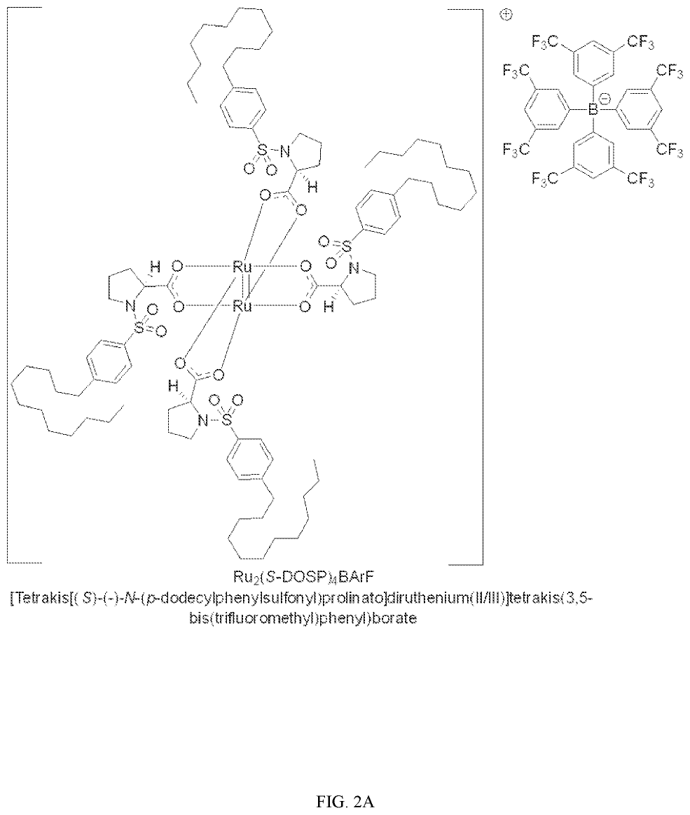

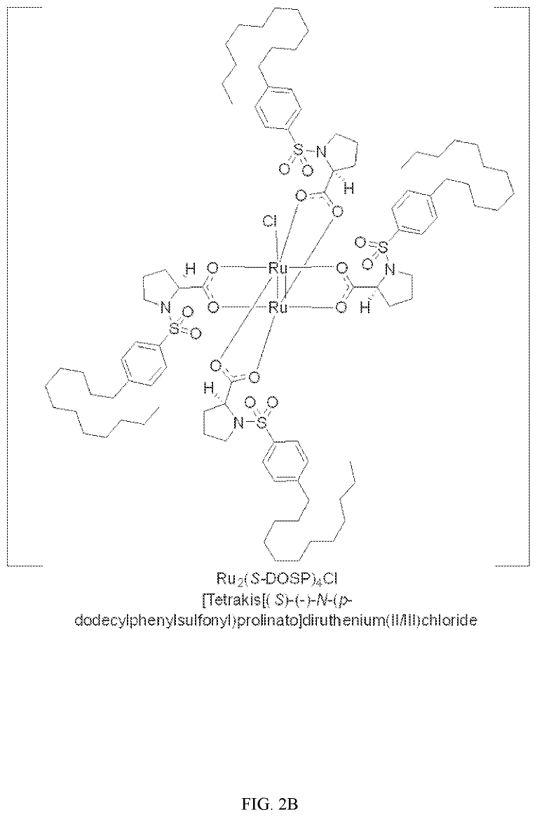

### DETAILED DESCRIPTION

### Terms

Unless defined otherwise, all technical and scientific terms used herein have the same meaning as is commonly understood by one of skill in the art. The following definitions are provided to help interpret the disclosure and claims of this application. In the event a definition in this section is not consistent with definitions elsewhere, the definition set forth in this section will control.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group, or visa versa. Derivatives may be prepared by any variety of synthetic methods or appropriate adaptations presented in synthetic or organic chemistry textbooks, such as those provide in March's Advanced Organic Chemistry: Reactions,

Mechanisms, and Structure, Wiley, 6th Edition (2007) Michael B. Smith or Domino Reactions in Organic Synthesis, Wiley (2006) Lutz F. Tietze, hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

When used in reference to compound(s) disclosed herein, “salts” refer to derivatives of the disclosed compound(s) where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or “heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” or “Ar” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above attached through a sulfamoyl bridge (i.e., —S(═O)₂NHalkyl), and an “Arylsulfamoyl” refers to an alkyl attached through a sulfamoyl bridge (i.e., —S(═O)₂NHaryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

### Diruthenium Catalysts

In certain embodiments, the disclosure relates to a diruthenium catalyst comprising a cyclopropyl ring substituted with a carboxylic acid ligand.

In certain embodiments, the disclosure relates to compositions comprising a diruthenium complex of the following formula,

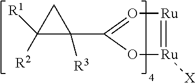

derivatives or salts thereof wherein,

X is a covalently bound ligand, such as Cl or other halogen, or a counterion such as PF₅⁻ or BARF⁻;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkyl sulfonyl, arylsulfonyl, carbocyclyl, aryl, or heterocyclyl, wherein R² is optionally substituted with one or more, the same or different, R⁴;

R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkyl sulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkyl sulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethysulfamoyl, carbocyclyl, aryl, or heterocyclyl.

In certain embodiments, the disclosure relates to compositions comprising a diruthenium complex of the following formula,

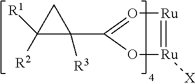

derivative or salt thereof wherein,

X is a ligand or a counter anion;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;

R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the counter anion is tetrakis(3,5-bis(trifluoromethyl)phenyl)borate (BArF).

In certain embodiments, the ligand is a halogen or chlorine (Cl).

In certain embodiments, the diruthenium complex is tetrakis [1-(4-phenyl(phenyl))-2,2-diphenylcyclopropanecarboxylato]diruthenium.

In certain embodiments, the diruthenium complex is tetrakis[1-(5-bromo-2-chlorophenyl)-2,2-diphenylcyclopropanecarboxylato]diruthenium.

In certain embodiments, the diruthenium complex is tetrakis [1,2,2-tris[4′-(tert-butyl)-(1,1′-biphenyl]-4-yl]cyclopropane-1-carboxylato]diruthenium].

In certain embodiments, the diruthenium complex is tetrakis [1-(4, 4″-di-tert-butyl-[1,1′:3′,1″-terphenyl]-5′-yl)-2,2-diphenylcyclopropanecarboxylato] diruthenium.

In certain embodiments, the diruthenium complex is tetrakis[1-(4-bromophenyl)-2,2-diphenylcyclopropanecarboxylato]diruthenium.

In certain embodiments, the diruthenium complex is in a composition with greater than 55%, 75%, 85%, 95%, 98%, or 99% enantiomeric excess of the following stereoisomers:

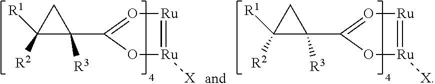

In certain embodiments, R¹ is carbocyclyl, aryl, or heterocyclyl.

In certain embodiments, R¹ is an aryl or an aromatic heterocyclyl.

In certain embodiments, R¹ is a phenyl optionally substituted with one or more R⁴.

In certain embodiments, R¹ is a phenyl optionally substituted with one or more halogen, alkyl, or alkoxy.

In certain embodiments, R¹ is hydrogen.

In certain embodiments, R² is carbocyclyl, aryl, or heterocyclyl.

In certain embodiments, R² is an aryl or an aromatic heterocyclyl.

In certain embodiments, R² is a phenyl optionally substituted with one or more R⁴.

In certain embodiments, R² is a phenyl optionally substituted with one or more halogen, alkyl, or alkoxy.

In certain embodiments, R³ is carbocyclyl, aryl, or heterocyclyl.

In certain embodiments, R³ is an aryl or an aromatic heterocyclyl.

In certain embodiments, R³ is a phenyl optionally substituted with one or more R⁴.

In certain embodiments, R³ is a phenyl optionally substituted with one or more halogen, alkyl, or alkoxy.

In certain embodiments, R³ is a phenyl substituted with a halogen.

In certain embodiments, R³ is a phenyl substituted with a halogen in the otho or para position.

In certain embodiments, the disclosure relates to compositions comprising a diruthenium complex of the following formula,

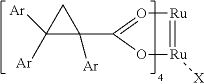

wherein X is a covalently bound ligand, such as a halogen, Cl, or a counterion such as PF₅⁻ or BARF⁻.

In certain embodiments, the disclosure relates to compositions comprising a diruthenium complex of the following formula,

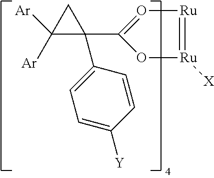

wherein X is a covalently bound ligand, such as a halogen, Cl, or a counterion such as PF₅⁻ or BARF⁻ and

Y is an electron withdrawing group.

In certain embodiments, the catalyst is in a composition with greater than 55%, 75%, 85%, 95%, 98%, or 99% enantiomeric excess of the following stereoisomers:

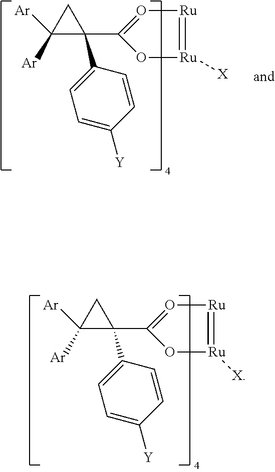

In certain embodiments, Y is a halogen.

In certain embodiments, the disclosure relates to compositions comprising a diruthenium complex of the following formula,

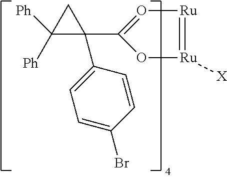

wherein X is a covalently bound ligand, such as a halogen, Cl, or a counterion such as PF₅⁻ or BARF⁻.

In certain embodiments, the disclosure relates to compositions comprising a diruthenium complex of the following formula,

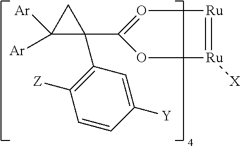

wherein X is a covalently bound ligand, such as a halogen, Cl, or a counterion such as PF₅⁻ or BARF⁻;

wherein Y is an electron withdrawing group; and

wherein Z is an electron withdrawing group.

In certain embodiments, Y is a halogen.

In certain embodiments, Z is a halogen.

In certain embodiments, the catalyst is in a composition with greater than 55%, 75%, 85%, 95%, 98%, or 99% enantiomeric excess of the following stereoisomers:

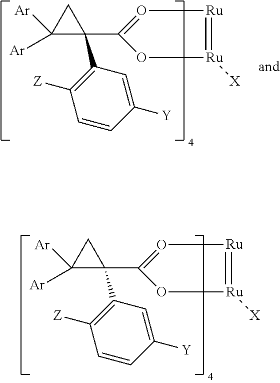

wherein X is a covalently bound ligand, such as a halogen, Cl, or a counterion such as PF₅⁻ or BARF⁻;

wherein Y is an electron withdrawing group; and

wherein Z is an electron withdrawing group.

In certain embodiments, Y is a halogen.

In certain embodiments, Z is a halogen.

In certain embodiments, the disclosure relates to compositions comprising a diruthenium complex of the following formula,

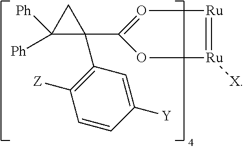

wherein X is a covalently bound ligand, such as a halogen, Cl, or a counterion such as PF₅⁻ or BARF⁻;

wherein Y is a halogen; and

wherein Z is a halogen.

In certain embodiments, the catalyst is in a composition with greater than 55%, 75%, 85%, 95%, 98%, or 99% enantiomeric excess of the following stereoisomers:

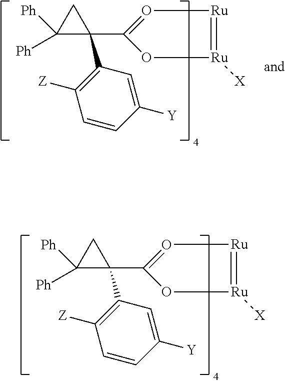

wherein X is a covalently bound ligand, such as a halogen, Cl, or a counterion such as PF₅⁻ or BARF⁻;⁻

wherein Y is a halogen; and

wherein Z is a halogen.

In certain embodiments, Y is a halogen.

In certain embodiments, Z is a halogen.

In certain embodiments, Y is a Br.

In certain embodiments, Z is a Cl.

In certain embodiments, the disclosure relates to a diruthenium catalyst comprising a pyrrolidine-2-sulfonamide substituted with a carboxylic acid ligand.

In certain embodiments, the disclosure relates to compositions comprising Ru₂(R-DOSP)₄ X, or a diruthenium complex of the following formula of formula

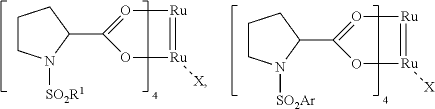

or derivatives or salts thereof wherein,

wherein X is a covalently bound ligand, such as a halogen, Cl, or a counterion such as PF₅⁻ or BARF⁻;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, Ar is p-alkyl-C₆H_{4—.}

In certain embodiments, this disclosure relates to compositions comprising a diruthenium complex of the following formula,

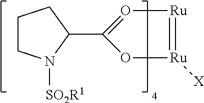

or salt thereof wherein,

X is a ligand or a counter anion;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the counter anion is tetrakis(3,5-bis(trifluoromethyl)phenyl)borate (BArF).

In certain embodiments, the ligand is a halogen or chlorine (Cl).

In certain embodiments, R¹ is (p-alkylphenyl).

In certain embodiments, the diruthenium complex is tetrakis[N-(p-dodecylphenylsulfonyl) prolinato]diruthenium.

In certain embodiments, the disclosure relates to compositions comprising Ru₂(S-TCPTTL)₄ X, Ru₂(S-PTAD)₄ X, Ru₂(S-TCPTAD)₄ X or a diruthenium complex of the following formula of formula

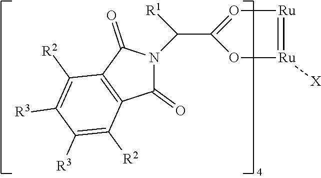

or derivatives or salts thereof wherein, wherein X is a covalently bound ligand, such as a halogen, Cl, or a counterion such as PF₅⁻ or BARF⁻;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;

R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcaramoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R² is hydrogen.

In certain embodiments, R³ is hydrogen.

In certain embodiments, R² and R³ are hydrogen.

In certain embodiments, R¹ is tertbutyl.

In certain embodiments, R¹ is a carbocyclyl.

In certain embodiments, R¹ is adamantly.

In certain embodiments, R² is phenyl.

In certain embodiments, R³ is phenyl.

In certain embodiments, R² and R³ are phenyl.

In certain embodiments, R² is halogen.

In certain embodiments, R³ is halogen.

In certain embodiments, R² and R³ are halogen.

In certain embodiments, this disclosure relates to composition comprising a diruthenium complex of the following formula,

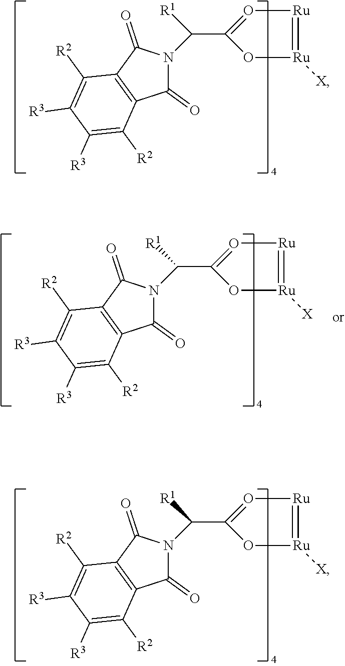

derivative or salt thereof wherein,

X is a ligand or a counter anion;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;

R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the counter anion is tetrakis(3,5-bis (trifluoromethyl)phenyl)borate (BArF).

In certain embodiments, the ligand is a halogen or chlorine (Cl).

In certain embodiments, R¹ is a carbocyclyl or is adamantly.

In certain embodiments, the diruthenium complex is tetrakis [2-(1,3-dioxo-4,5,6,7-tetraphenylisoindolin-2-yl)-3,3-dimethylbutanato]diruthenium.

In certain embodiments, the diruthenium complex is tetrakis[(1-adamantyl)-(N-tetrachlorophthalimido)acetato]diruthenium.

In certain embodiments, the diruthenium complex is tetrakis[(1-adamantyl)-(N-phthalimido)acetato]diruthenium.

### Synthetic Processes

Many enantioselective reactions of donor/acceptor carbenoids may be utilized with diruthenium catalysts disclosed herein. In certain embodiments, the diruthenium complex can be used to make intermediates for pharmaceutical agents as exemplified herein. See Davies & Manning, Nature, 2008, 451, 417-424; Davies & Denton, Chem. Soc. Rev., 2009, 38, 3061-3071; Davies, et al., J. Am. Chem. Soc., 2006, 128, 2485-2490; and Davies &. Walji, Angew. Chem., Int. Ed., 2005, 44, 1733-1735, all hereby incorporated by reference.

In certain embodiments, the disclosure contemplates reactions of donor/acceptor carbenoids, such as cyclopropanations, formal [4+3] cycloadditions, C—H functionalizations, and ylide transformations comprising mixing a compound comprising a carbenoid precursor, e.g., a diazo compound, and catalysts disclosed herein and reactive compounds under conditions such that a synthetic compound is formed.

In certain embodiments, the disclosure relates to methods of making a synthetic compound comprising mixing a) a diazo compound, b) a compound with a carbon hydrogen bond, and c) a diruthenium catalyst disclosed herein, under conditions such that a synthetic compound is formed comprising a carbon-to-carbon bond between the diazo compound and the compound with a carbon hydrogen bond. The synthetic compound may be the result of an inter or an intra-molecular reaction.

In certain embodiments, the disclosure relates to methods of making a synthetic compound comprising mixing a) a diazo compound, b) a compound with a nitrogen-to-hydrogen bond, and c) a diruthenium catalyst disclosed herein, under conditions such that a synthetic compound is formed comprising a carbon-to-nitrogen bond between the diazo compound and the compound with a nitrogen hydrogen bond. The synthetic compound may be the result of an inter or an intra-molecular reaction.

In certain embodiments, the disclosure relates to cyclopropanation reactions using catalysts disclosed herein. See Davies et al., J. Am. Chem. Soc. 1996, 118, 6897-6907, hereby incorporated by reference. In certain embodiments, the disclosure relates to methods of making a synthetic compound comprising mixing a) a diazo compound such as vinyldiazomethane and vinyldiazoactates optionally substituted with one or more substituents, b) a double bond compound such as an alkene or diene optionally substituted with one or more substituent, and c) a compound comprising a diruthenium catalyst disclosed herein under conditions such that a synthetic compound is formed comprising a cyclopropyl ring and carbon-to-carbon bond between the diazo compound and the double bond compound.

In certain embodiments, the diazo compound has the following formula,

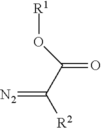

the double bond compound has the following formula,

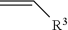

and the synthetic compound has the following formula,

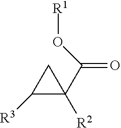

wherein, R¹ is alkyl, carbocyclyl, aryl, or heterocyclyl, wherein R¹ is optionally substituted with one or more, the same or different, R⁴;

R² is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;

R³ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ is alkyl optionally substituted with one or more, the same or different, R⁴.

In certain embodiments, R² is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁴.

In certain embodiments, R² is aryl or an aromatic heterocyclyl optionally substituted with one or more, the same or different, R⁴.

In certain embodiments, R³ is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁴.

In certain embodiments, R³ is aryl or an aromatic heterocyclyl optionally substituted with one or more, the same or different, R⁴.

In certain embodiments, the synthetic product is in a composition with greater than 55%, 75%, 85%, 95%, 98%, or 99% enantiomeric excess of the following stereoisomers:

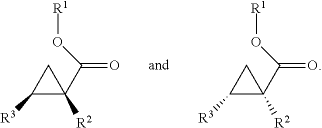

In certain embodiments, the diazo compound has the following formula,

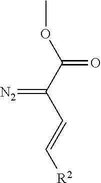

the double bond compound has the following formula,

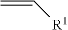

and the synthetic compound has the following formula,

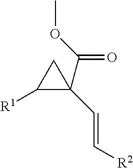

or salts thereof wherein

R¹ is phenyl optionally substituted with one or more substituent and

R² is phenyl optionally substituted with one or more substituent.

In certain embodiments, the synthetic product is in a composition with greater than 55%, 75%, 85%, 95%, 98%, or 99% enantiomeric excess of the following stereoisomers:

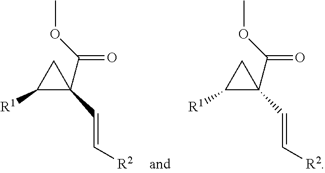

In certain embodiments, the diazo compound has the following formula,

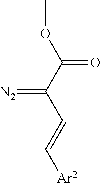

the double bond compound has the following formula,

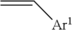

and the synthetic compound has the following formula,

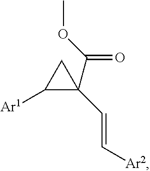

wherein Ar¹ and Ar² are each, the same or different, aryl optionally substituted with one or more sub stituents.

In certain embodiments, the synthetic product is in a composition with greater than 55%, 75%, 85%, 95%, 98%, or 99% enantiomeric excess of the following stereioisomers:

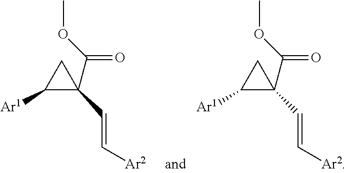

In certain embodiments, the disclosure contemplates the use of diruthenium catalysts disclosed herein in [4+3] cycloaddition reactions, e.g., mixing a diene and a vinyldiazoacetate and a diruthenium catalyst disclosed herein under conditions such that a cyclic compound is formed. See Davies et al, J. Am. Chem. Soc. 1998, 120, 3326-3331 and Reddy & Davies, J. Am. Chem. Soc., 2007, 129 (34), 10312-10313.

In certain embodiments, the diazo compound is a vinyldiazoacetate of the following formula,

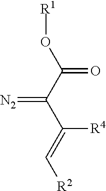

the diene compound has the following formula,

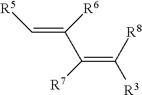

and the synthetic compound has the following formula,

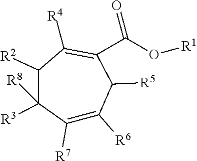

wherein, R¹ is alkyl, carbocyclyl, aryl, or heterocyclyl, wherein R¹ is optionally substituted with one or more, the same or different, R⁹;

R² is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁹;

R³ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁹;

R⁴ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁹;

R⁵ is hydrogen, alkyl, halogen, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁹;

R⁶ is hydrogen, alkyl, halogen, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁶ is optionally substituted with one or more, the same or different, R⁹;

R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁷ optionally substituted with one or more, the same or different, R⁹;

R⁸ is hydrogen, alkyl, halogen, cyano, hydroxy, amino, mercapto, formyl, carboxy, carbamoyl, alkoxy, alkanoyl, alkylthio, alkylamino, (alkyl)₂amino, alkyl sulfinyl, alkylsulfonyl, arylsulfonyl, carbocyclyl, aryl, or heterocyclyl, wherein R⁸ is optionally substituted with one or more, the same or different, R⁹;

R⁹ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁹ is optionally substituted with one or more, the same or different, R¹⁰; and

R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the synthetic product is in a composition with greater than 55%, 75%, 85%, 95%, 98%, or 99% enantiomeric excess of the following stereoisomers:

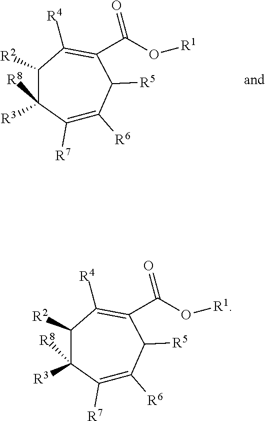

In certain embodiments, R¹ is alkyl optionally substituted with one or more, the same or different, R⁹.

In certain embodiments, R² is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁹.

In certain embodiments, R² is aryl or an aromatic heterocyclyl optionally substituted with one or more, the same or different, R⁹.

In certain embodiments, R³ is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁹.

In certain embodiments, R³ is aryl or an aromatic heterocyclyl optionally substituted with one or more, the same or different, R⁹.

In certain embodiments, R⁴ is hydrogen.

In certain embodiments, R⁵ is hydrogen.

In certain embodiments, R⁶ is hydrogen.

In certain embodiments, R⁷ is hydrogen.

In certain embodiments, R⁸ is hydrogen.

In certain embodiments, the diazo compound is a vinyldiazoacetate of the following formula,

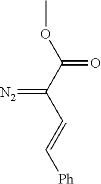

the diene compound has the following formula,

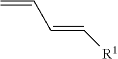

and the synthetic compound has the following formula,

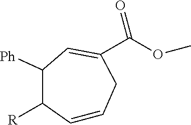

Wherein R is phenyl optionally substituted with one or more substituent.

In certain embodiments, the synthetic product is in a composition with greater than 55%, 75%, 85%, 95%, 98%, or 99% enantiomeric excess of the following stereioisomers:

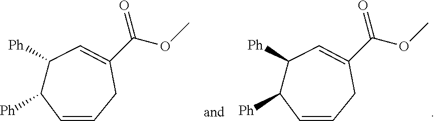 [SMILES: COC(=O)C1=C[C@@H](c2ccccc2)[C@@H](c2ccccc2)C=CC1 ; COC(=O)C1=C[C@H](c2ccccc2)[C@H](c2ccccc2)C=CC1]

In certain embodiments, the diazo compound has the following formula,

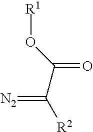

the compound has the following formula,

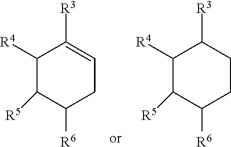

and the synthetic compound has the following formula,

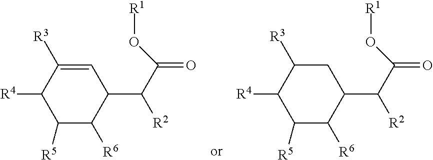

wherein, R¹ is alkyl, carbocyclyl, aryl, or heterocyclyl, wherein R¹ is optionally substituted with one or more, the same or different, R⁷;

R² is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁷;

R³ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁷;

R⁴ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁷;

R⁵ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁷; or R⁴ and R⁵ form a ring selected from a carbocyclyl, aryl, or heterocyclyl wherein the ring is optionally substituted with one or more, the same or different, R⁷

R⁶ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁶ is optionally substituted with one or more, the same or different, R⁷;

R⁷ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸;

R⁸ is alkyl, halogen, cyano, hydroxy, amino, mercapto, formyl, carboxy, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁸ is optionally substituted with one or more, the same or different, R⁹; and

R⁹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ is alkyl optionally substituted with one or more, the same or different, R⁷.

In certain embodiments, R² is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁷.

In certain embodiments, R² is aryl or an aromatic heterocyclyl optionally substituted with one or more, the same or different, R⁷.

In certain embodiments, R³ is an electron donating group selected from cyano, alkenyl, alkynyl, formyl, carbamoyl, carboxy, alkylsulfinyl, alkylsulfonyl, or arylsulfonyl, aryl, or an aromatic heterocyclyl optionally substituted with one or more, the same or different, R⁷.

In certain embodiments, R³ is hydrogen or alkyl optionally substituted with one or more, the same or different, R⁷.

In certain embodiments, the disclosure relates to diazo compound of the following formula:

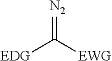

wherein EDG is an electron withdrawing group and EWG is an electron donating group. Such a diazo compound may react with a compound with a C—H bond or a N—H bond as further exemplified below. See Davies & Morton, Chem. Soc. Rev., 2011, 40, 1857-1869, hereby incorporated by reference.

In certain embodiments, the diazo compound has the following formula,

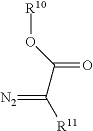

R¹⁰ is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹²;

R¹¹ is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹² is optionally substituted with one or more, the same or different, R⁹;

R¹² is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹² is optionally substituted with one or more, the same or different, R¹³;

R¹³ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetyl amino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl -N-ethylsulfamoyl, carbocyclyl, aryl, or heterocyclyl.

In certain embodiments, R¹⁰ is alkyl optionally substituted with one or more halogens.

In certain embodiments, R¹¹ is aryl or phenyl optionally substituted with an ortho or para substituted with one or more a halogen, hydroxy, alkoxy, thiol, alkylthio, amino, or alkylamino.

In certain embodiments, the method comprises mixing the diazo compound with a compound of the following formula:

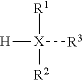

under conditions such that a synthetic compound of the following formula is formed,

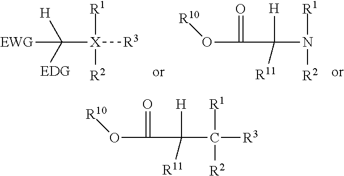

wherein X is carbon or nitrogen, wherein R³ is absent if X is nitrogen,

R¹ is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;

R² is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;

or R¹ and R² form a carbocyclyl, aryl, or heterocyclyl ring optionally substituted with one or more, the same or different, R⁴; or when X is carbon R¹, R², and R³ form a multicyclic carbocyclyl;

R³ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and

R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl;

R¹⁰ is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹²;

R¹¹ is alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹²;

R¹² is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹² is optionally substituted with one or more, the same or different, R¹³;

R¹³ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the disclosure contemplates the use of diruthenium catalysts disclosed herein for the intra-molecular insertion reactions for the formation of carbon-to-carbon bonds. In certain embodiment, disclosure contemplates a method of making a ring structure comprising mixing a diruthenium catalyst disclosed herein and a compound comprising a diazo group and the same compound comprising a C-H bond configure about the molecule to form a five or six membered ring.

In certain embodiments, the disclosure contemplates the use of diruthenium catalysts disclosed herein in C—H aminations. In certain embodiments, the disclosure contemplates the use of diruthenium catalysts disclosed herein for the insertion reactions for the formation of carbon to nitrogen bonds, e.g., inter and intra-molecular C—H aminations. See Reddy & Davies, Org. Lett., 2006, 8, 5013-5016, hereby incorporated by reference.

In certain embodiments, the disclosure relates to a method of making a synthetic compound comprising mixing a compound with an aromatic compound, PhI(OAc)₂, NsNH₂ (4-nitrobenzenesulfonamide, and a diruthenium catalysts disclosed herein under conditions such that a synthetic compound with a C—N bond is formed.

In certain embodiments, the aromatic compound is a has the following formula

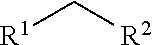

and the synthetic compound is

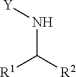

wherein, Y is 4-nitrobenzenesulfonamide or 2-nitrobenzenesulfonamide;

R¹ is aryl or aromatic heterocyclyl wherein le is optionally substituted with one or more, the same or different, R³;

R² is hydrogen alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R³;

or R¹ and R² together with the attached atoms form a carbocyclyl, aryl, or heterocyclyl optionally substituted with one or more, the same or different, R³;

R³ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;

R⁴ is hydrogen, alkyl, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;

R⁵ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, the synthetic product is in a composition with greater than 55%, 75%, 85%, 95%, 98%, or 99% enantiomeric excess of the following stereioisomers:

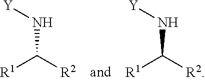

In certain embodiments, the disclosure relates to a method of making Rasagiline comprising the step of mixing 2,3-dihydro-1H-indene, PhI(OAc)₂, YNH₂, MgO, and a diruthenium catalyst disclosed herein, wherein Y is 4-nitrobenzenesulfonamide or 2-nitrobenzenesulfonamide, under conditions such that a compound of the following formula is formed,

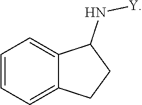

Reaction of this product with propargyl bromide in potassium carbonate provides the N substituted propargyl product. Removal of the nitrobenzenesulfonamide with DBU and HSCH₂CH₂OH provides Rasagiline. See Reddy & Davies, Org. Lett., 2006, 8, 5013-5016, hereby incorporated by reference.

### Experimental

The following is intended to provide examples on methods of making and using embodiments of the disclosure. It is not intended to limit the scope.

### Synthesis and Characterization of a Diruthenium Catalysts

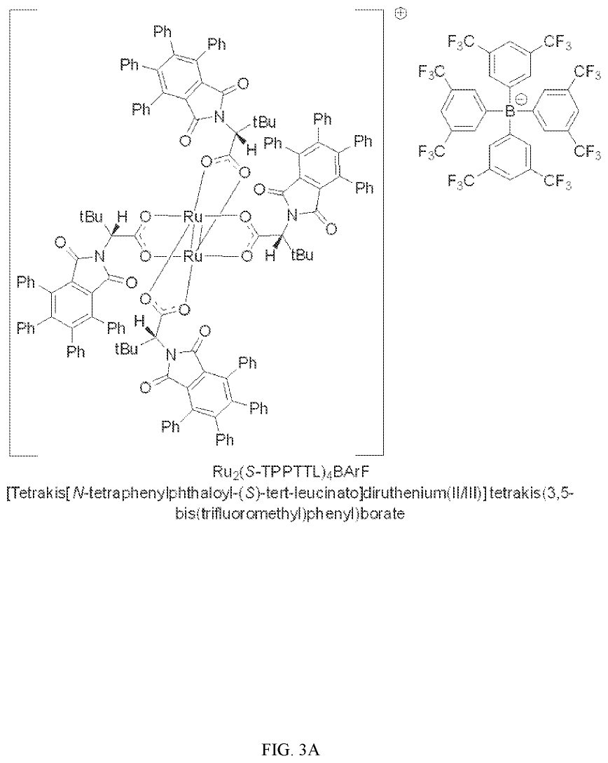

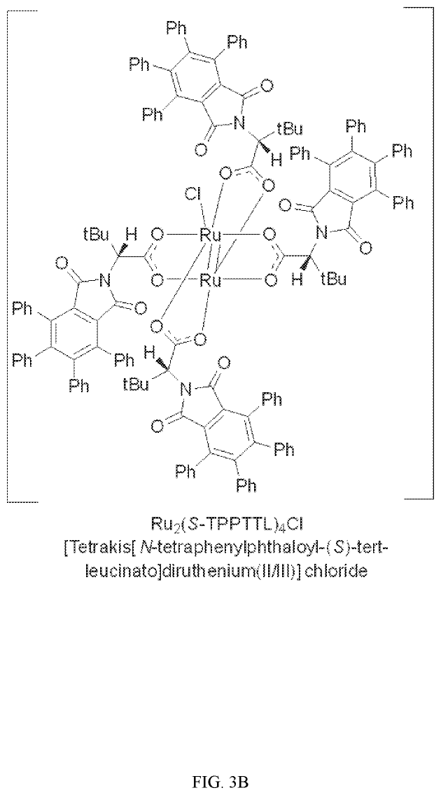

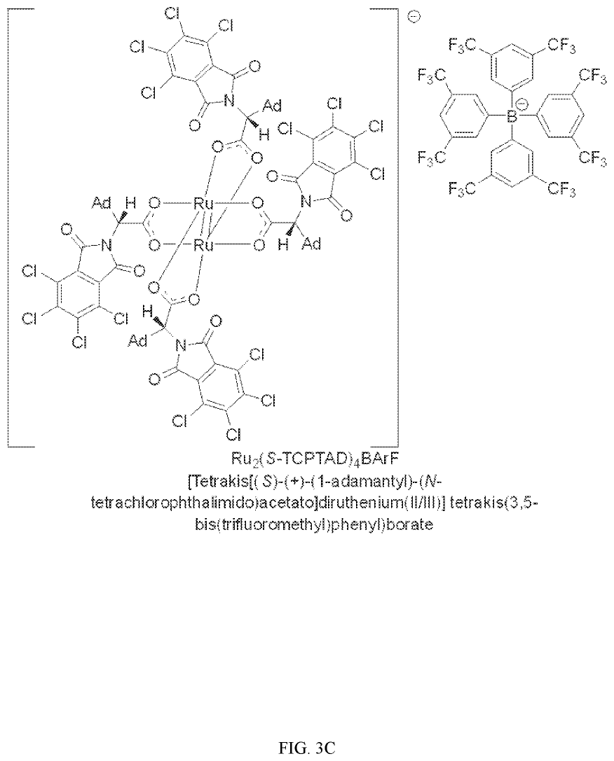

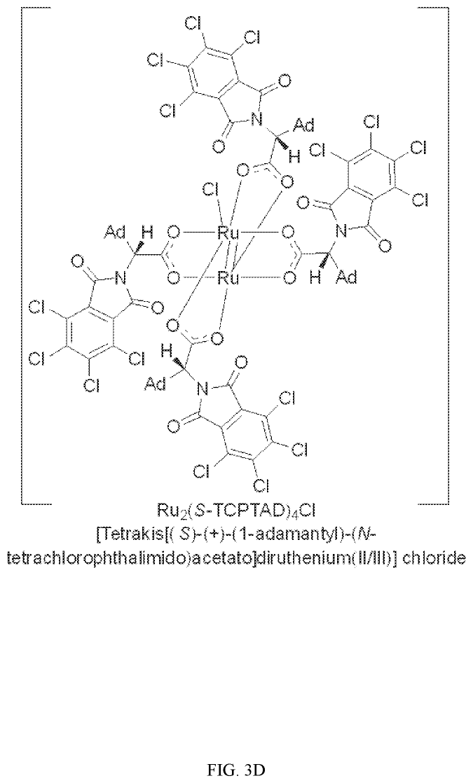

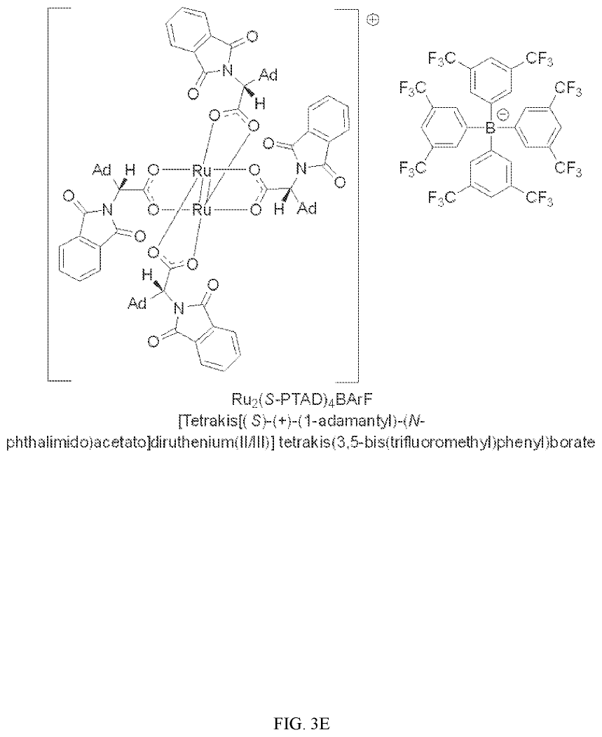

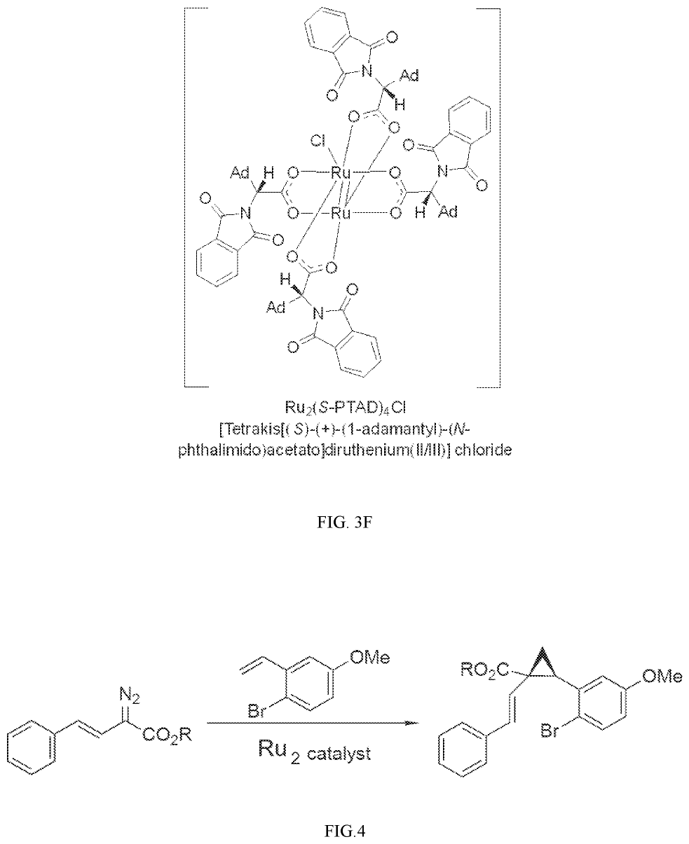

To confirm that the ligands self-assemble to generate the symmetry structures of diruthenium complexes studies were performed with a S-TPPTTL ligand (FIG. 3B). Heating the ligand with Ru₂(OAc)₄Cl, results in the formation of a brown solid, that was confirmed by mass-spectrometry to be the desired diruthenium complex. Treatment of Ru₂(S-TPPTTL)₄Cl with NaBArF, displaces the ruthenium bound chloride and generates the diruthenium cationic species [Ru₂(S-TPPTTL)₄]⁺ BARF⁻. The X-ray structure of Ru₂(TPPTTL)₄Cl showed that the complex had adopted a C₄-symmetric bowl-shaped structure.

### Enantioselective Cyclopropanation with Donor/Acceptor Carbenes

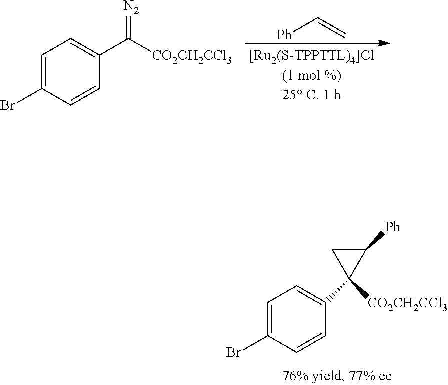

Results with diazomalonate gave moderate results (44% yield, 46% ee) because the reactions were conducted at 60° C. However, aryldiazoacetates are more responsive to certain catalysts. As a test reaction a standard cyclopropanation reaction between an aryldiazoacetate and styrene was conducted. The reaction with Ru₂(S-TPPTTL)₄Cl was be conducted at room temperature and generated the cyclopropane in 76% yield and 77% ee.

### Enantioselective C—H Functionalization

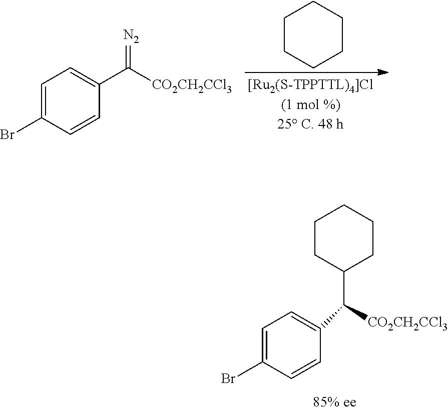

A reaction of the aryldiazoacetate with cyclohexane was performed. Cyclohexane is a challenging substate and because it is 28,000 times less reactive that styrene in certain reactions with donor/acceptor carbenes. It was observed that cyclohexane was an effective trap, generating the C—H functionalized product in 85% ee. Even more impressive is the reaction with [Ru₂(TPPTTL)₄]+ BArF− as catalyst at 25° C., which generated the C—H functionalization product in 76% yield and 95% ee.

What is claimed is:

1. A composition comprising a diruthenium complex of the following formula, 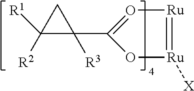
or salt thereof wherein,
X is a ligand or a counter anion;
R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;
R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;
R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;
R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;
R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and
R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

2. The composition of claim 1 wherein the counter anion is tetrakis(3,5-bis (trifluoromethyl)phenyl)borate (BArF).

3. The composition of claim 1 wherein the ligand is a halogen or chlorine (Cl).

4. The composition of claim 1 wherein the diruthenium complex is tetrakis [1-(4-phenyl (phenyl))-2,2-diphenylcyclopropanecarboxylato]diruthenium.

5. The composition of claim 1 wherein the diruthenium complex is tetrakis[1-(5-bromo-2-chlorophenyl)-2,2-diphenylcyclopropanecarboxylato]diruthenium.

6. The composition of claim 1 wherein the diruthenium complex is tetrakis [1,2,2-tris[4′-(tert-butyl)-(1,1′-biphenyl]-4-yl]cyclopropane-1-carboxylato]diruthenium].

7. The composition of claim 1 wherein the diruthenium complex is tetrakis [1-(4,4″-di-tert-butyl-[1,1′:3′,1″-terphenyl]-5′-yl)-2,2-diphenylcyclopropanecarboxylato]diruthenium.

8. The composition of claim 1 wherein the diruthenium complex is tetrakis[1-(4-bromophenyl)-2,2-diphenylcyclopropanecarboxylato]diruthenium.

9. A composition comprising a diruthenium complex of the following formula, 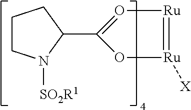
or salt thereof wherein,
X is a ligand or a counter anion;
R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;
R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;
R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and
R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

10. The composition of claim 9 wherein the counter anion is tetrakis (3,5-bis(trifluoromethyl)phenyl)borate (BArF).

11. The composition of claim 9 wherein the ligand is a halogen or chlorine (Cl).

12. The composition of claim 9 wherein R¹ is (p-alkylphenyl).

13. The composition of claim 9 wherein the diruthenium complex is tetrakis[N-(p-dodecylphenylsulfonyl)prolinato]diruthenium.

14. A composition comprising a diruthenium complex of the following formula, 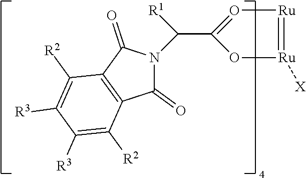
or salt thereof wherein,
X is a ligand or a counter anion;
R¹ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R¹ is optionally substituted with one or more, the same or different, R⁴;
R² is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R² is optionally substituted with one or more, the same or different, R⁴;
R³ is hydrogen, alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R³ is optionally substituted with one or more, the same or different, R⁴;
R⁴ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁴ is optionally substituted with one or more, the same or different, R⁵;
R⁵ is alkyl, halogen, cyano, hydroxy, amino, mercapto, formyl, carboxy, carbamoyl, alkoxy, alkanoyl, alkylthio, alkylamino, (alkyl)₂amino, alkylsulfinyl, alkylsulfonyl, arylsulfonyl, carbocyclyl, aryl, or heterocyclyl, wherein R⁵ is optionally substituted with one or more, the same or different, R⁶; and
R⁶ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

15. The composition of claim 14, wherein the counter anion is tetrakis(3,5-bis (trifluoromethyl)phenyl)borate (BArF).

16. The composition of claim 14, wherein the ligand is a halogen or chlorine (Cl).

17. The composition of claim 14, wherein R¹ is a carbocyclyl or is adamantly.

18. The composition of claim 14, wherein the diruthenium complex is tetrakis[2-(1,3-dioxo-4,5,6,7-tetraphenylisoindolin-2-yl)-3,3-dimethylbutanato]diruthenium.

19. The composition of claim 14, wherein the diruthenium complex is tetrakis[(1-adamantyl)-(N-tetrachlorophthalimido)acetato]diruthenium.

20. The composition of claim 14, wherein the diruthenium complex is tetrakis[(1-adamantyl)-(N-phthalimido)acetato]diruthenium.

21. A method of performing a synthetic transformation comprising contacting a diruthenium complex of claim 1 with a staring material providing a coupling product.
